(19) United States
(12) Patent Application Publication
(10) Pub. No.: US 2016/0262838 A1
(43) Pub. Date: Sep. 15, 2016

(54) SYSTEM FOR SURGICAL TRACKING

(71) Applicant: STRYKER GLOBAL TECHNOLOGY CENTER, Haryana (IN)

(72) Inventors: Ketan Jajal, Gujarat (IN); Sourab Dhami, Faridabad (IN); Subhashree Dipika Mohanty, Odisha (IN); Vivek Sharma, Haryana (IN)

(73) Assignee: Stryker Global Technology Center, Haryana (IN)

(21) Appl. No.: 15/036,358

(22) PCT Filed: Nov. 13, 2014
(86) PCT No.: PCT/IB2014/066012

§ 371 Date: May 12, 2016

(30) Foreign Application Priority Data
Nov. 13, 2013 (IN) 3345/DEL/2013

(51) Int. Cl. A61B 34/20

(52) U.S. Cl. CPC A61B 34/20; A61B 2034/2068; A61B 2034/2055

(57) ABSTRACT

Disclosed herein are systems for surgical tracking of predetermined target in a reference object. The systems comprise a surgical tracking unit and at least one tracker. The tracker comprises at least one tip to identify at least one target point of reference object, at least two different tracking patterns one being unrecognizable to the surgical tracking unit, and another being recognizable by the surgical tracking unit. Only one tracking pattern is exposed to the surgical tracking unit at any instant of time. An inbuilt mechanism has at least one trigger. The tip of the tracker contacts the at least one target point and a trigger is activated thereby altering the tracking pattern into a new tracking pattern which is recognizable by the surgical tracking unit thereby providing input of the target point of the reference object at any instant to the surgical tracking unit.

### FIELD OF THE INVENTION

The present invention relates to systems for surgical tracking. More particularly the invention discloses a system for surgical tracking of predetermined target in a reference object using a novel tracker.

### BACKGROUND OF THE INVENTION

The success of a surgical procedure generally lies on various factors which include minimum invasiveness and accurate positioning of the surgical instruments and implants with respect to the patient anatomy. In order to achieve accuracy during surgery, a surgeon often takes the assistance of surgical navigation systems.

Surgical navigation involves real time computerized monitoring of the spatial position as well as orientation of the tracking elements around implantation site with respect to certain key anatomical landmarks during surgery. The minimum invasiveness and ability to correctly pinpoint the position of the surgical object with respect to the patient at any instant of time are the hallmark of these surgical navigation systems.

Surgical navigation systems generally comprise computer systems which include sensors to detect, track or monitor the tracking elements attached in predetermined relationship with regard to the surgical site. Surgical navigation system usually employs markers which are fixed on the surgical instruments as a reference marker in order to ascertain their position in the complex background of the implantation site. These markers may be passive markers which are essentially reflecting or retro-reflecting markers, and active markers which include light emitting markers, or magnetic markers which are constantly sending information to the main system.

The advantage of an active marker over the passive marker is that it allows communication directly from the sterile area to capture discrete point or area of an anatomy without the need of an additional person at the computer station, outside the sterile field, for example, to manually accept the comment and feed the input.

The state-of-art tracking technologies employing active tracking as described in U.S. Pat. Pub. No. 2001/0034530 the disclosure of which is hereby incorporated by reference, typically uses infra-red or other non-visible optical tracking technology. However, active tracking requires power inputs typically a sterile battery which adds cost and complexities to the surgery.

U.S. Pat. Pub. No. 2011/0254922 discloses a system for detecting a position of an object such as a surgical tool in an image guidance system which includes a camera system with a detection array for detecting visible light, a processor arranged to analyze the output from the array, the disclosure of which is hereby incorporated by reference. Each object to be detected carries a single marker with a pattern of contrasted areas of light and dark intersecting at a specific single feature point thereon. The pattern includes components arranged in an array around the specific location arranged such that the processor is able to detect an angle of rotation of the pattern around the location and which are different from other markers of the system such that the processor is able to distinguish each marker from the other markers.

The main disadvantage of this method is that the pattern of contrast is always visible to the camera system at all time. This causes a problem to the surgeon to identify and detect the trackers specific to the surgical target. There is neither provision to detect and input a discreet event nor there is possibility of providing input by the surgeon during surgery. This therefore requires an additional person to manually input the data to record the discrete position of the tracker.

### BRIEF SUMMARY OF THE INVENTION

In order to overcome the drawbacks of the current state of the art, the main object of the present invention is to provide a system for surgical tracking/navigation which is capable of identifying and capturing the desired or predetermined target point in a reference object such as position of the surgical object specific landmarks of patient anatomy and/or position of surgical instruments at any instant during surgical procedure.

Accordingly, the present invention provides a system for surgical tracking comprising a surgical tracking unit and trackers. The tracker may be a complex two-dimensional (“2D”) image sticker or may be a simple or complex image sticker or may be a representation of the image displayed on the electronic display screen. These trackers can be a multilayer image that can be modified via mechanical means to display a specific pattern at any given time in the surgery. The tracker can be of multiple layer sterile sheets, with a lower sticker sheet being opaque and an upper sheet partially transparent so as the lower sheet information can be revealed when the trigger is pressed.

The trackers can also be made of all metal or plastic such that when the lower and the upper layer are aligned at a specific location a pattern is generated that is recognizable by the tracking unit. The marking on these patterns can be made of, but not limited to, laser marking, embossing, window opening and could be of different colors for easy detection.

The trackers are also capable of taking inputs from the surgeon at any given time to record or capture a specific position at any moment during surgery.

These trackers may be passive trackers, wherein they have an inbuilt mechanism to change their image shape so that they may show a particular tracking pattern or tracker shape at any instant of time to identify the target which could have been lost in the image of neighboring tracker in the vicinity of target scheduled for surgery, this can be similar to but not limited to a Quick Response (“QR”) code image.

The surgical navigation/tracking unit is capable of identifying or recognizing the particular tracking pattern on the tracker. When that particular pattern appears on a tracker, the surgical tracking unit detects the target position which may be a landmark on anatomy or specific position of surgical instrument.

Accordingly the present invention relates to system for surgical tracking to identify the predetermined target point in the reference object. The system comprising of at least one surgical tracking unit and at least one tracker. The surgical tracking unit comprises of at least one sensor, at least one processing unit and at least one memory unit. The said system further comprises of tip to identify the specific target point of reference object. The tracker having at least two tracking pattern one being unrecognizable to surgical tracking unit, another being recognizable to the surgical tracking unit. At any instant only one pattern is exposed to surgical tracking unit. The tracker further has one inbuilt mechanism which is having at least one trigger. The inbuilt mechanism can alter or change the tracking pattern of the tracker. The said system further may have tip to identify the specific target point. The trigger provides input of the target point of reference object to the surgical tracking unit via inbuilt mechanism by altering tracking pattern to new tracking pattern which is recognizable to the sensor. Upon recognition, the surgical tracking unit captures the positional information of the tip of the tracker in its memory by determining the relative location of tip of the tracker and the sensor in its processing unit thereby enabling it to record at least one target point with in sterile environment during surgical procedure.

### BRIEF DESCRIPTION OF THE DRAWINGS

The foregoing objects, features, aspects and advantages will become more apparent from the following detailed description when taken in conjunction with the accompanying drawings, in which:

FIG. 1A shows the schematic illustration of a tracker as visible by the tracking unit in which the tracker pattern is such that it is not being detected by the tracking unit.

FIG. 1B shows the schematic illustration of the tracker of FIG. 1A as visible by the tracking unit in which the tracker pattern is such that it is being detected by the tracking unit and thereby the position of a landmark on patient's anatomy is captured by the tracking system.

FIG. 2A shows the schematic illustration of a tracker as visible by the tracking unit in which the tracker pattern is such that it is not being detected by the tracking unit.

FIG. 2B shows the schematic illustration of the tracker of FIG. 2A as visible by the tracking unit in which the tracker pattern is such that it is being detected by the tracking unit and thereby the position of a specific landmark on patient's anatomy is captured by the tracking unit.

FIG. 2C shows the schematic illustration of the tracker of FIG. 2A as visible by the tracking system in which the tracker pattern is such that it is being detected by the tracking unit and thereby the position of another landmark on patient's anatomy is captured by the same tracking unit.

### DETAILED DESCRIPTION

The present invention relates to a system for surgical tracking which comprises a surgical tracking unit and a tracker. The tracker can be used to accurately detect the surgical objects which may be an important reference of patient anatomy or may be a specific instrument position or position of an implant with reference to the anatomy of the patient during surgery.

The term “tracking pattern” is herein interchangeably referred to as a trackable element and/or pattern. The term “surgical navigation unit” is herein interchangeably referred to as surgical tracking unit or navigation unit or tracking unit. The term “trackers” is herein interchangeably referred to as “novel trackers” or tracking unit. “Reference object” is interchangeably referred to as surgical objects.

In a preferred embodiment, the present invention discloses a tracker having trackable element. The tracker is capable of being connected to an anatomical part of body of a patient. Further it can be attached to an instrument to be used by the surgeon during a surgical procedure as well as can act as an independent tracker to identify discrete landmarks of patient's anatomy. The trackers are passive and the surgeon may configure multiple said trackers to capture important information about patient's anatomy and/or implant system position information.

The trackers of the present invention may be similar in configuration and features. The trackers of the present invention has the capability of displaying a unique pattern which is recognizable to the tracking unit for a given time duration. This makes the trackers capable of communicating wirelessly to the tracking unit sensors without the use of any energy. Further, the trackers also have the capability of displaying multiple such patterns allowing it to communicate with the tracking unit for multiple anatomical landmarks of the patients or surgical instrument position.

In another embodiment the present invention may be used for various surgical procedures including but not limited to arthroplasty, sinus surgery, spinal surgery, trauma surgeries, brain surgery, as well as neurosurgery.

In yet another embodiment, the trackers of the present invention may also capture one or more patient position information during the surgery and use that information at a later state to accurately position the implant systems.

In yet another embodiment, the present invention may also be used for diagnostics either alone or in combination.

The process of the present invention may also be configured to be used for various diagnostic procedures.

The systems and the devices of the present invention can be easily placed in a sterile zone. Further, the systems and the devices of the present invention require minimal set up.

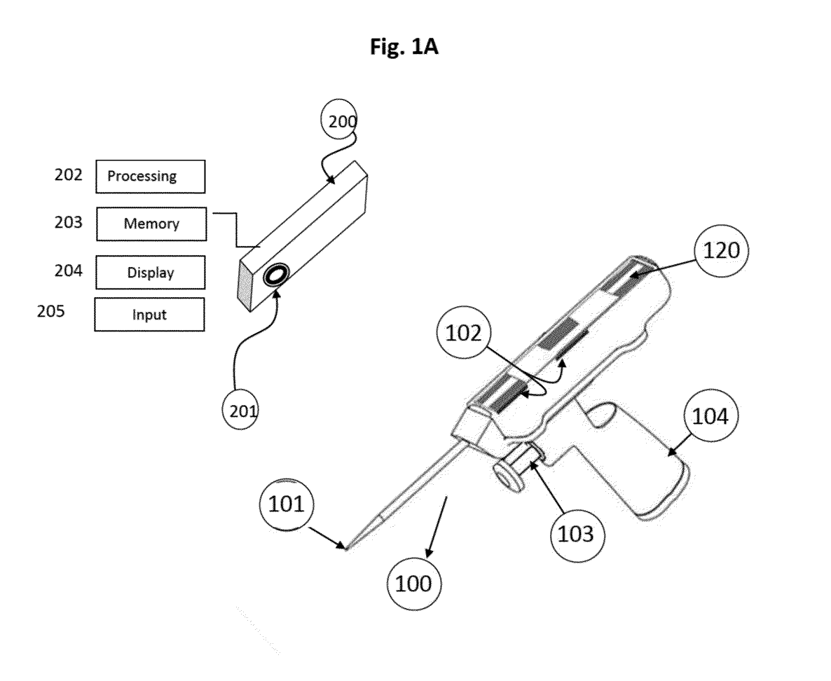

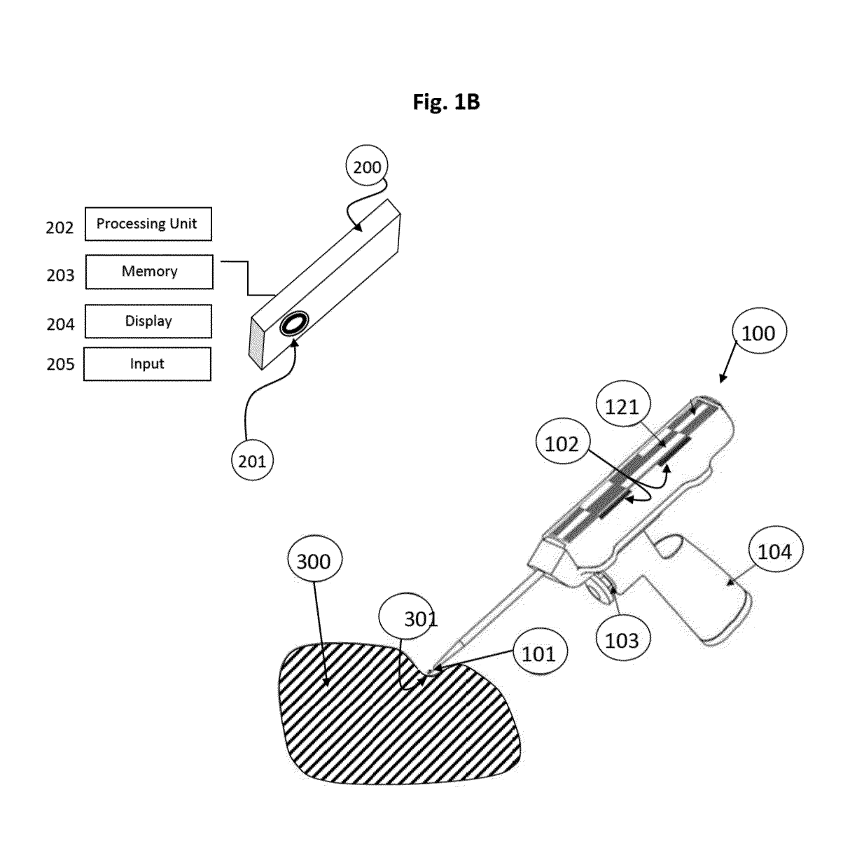

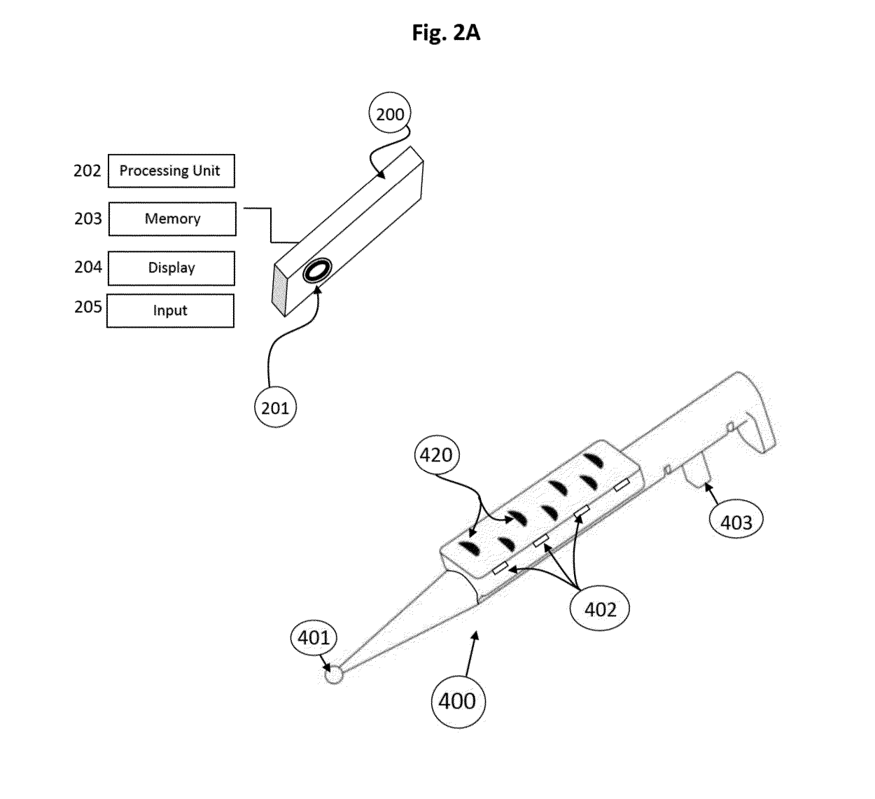

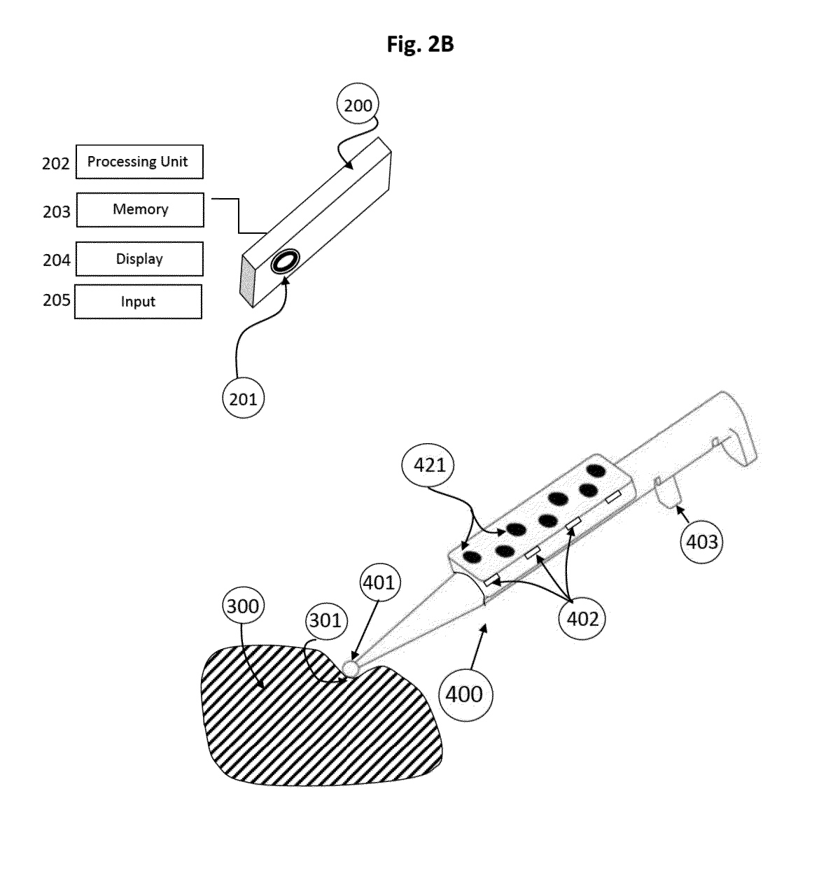

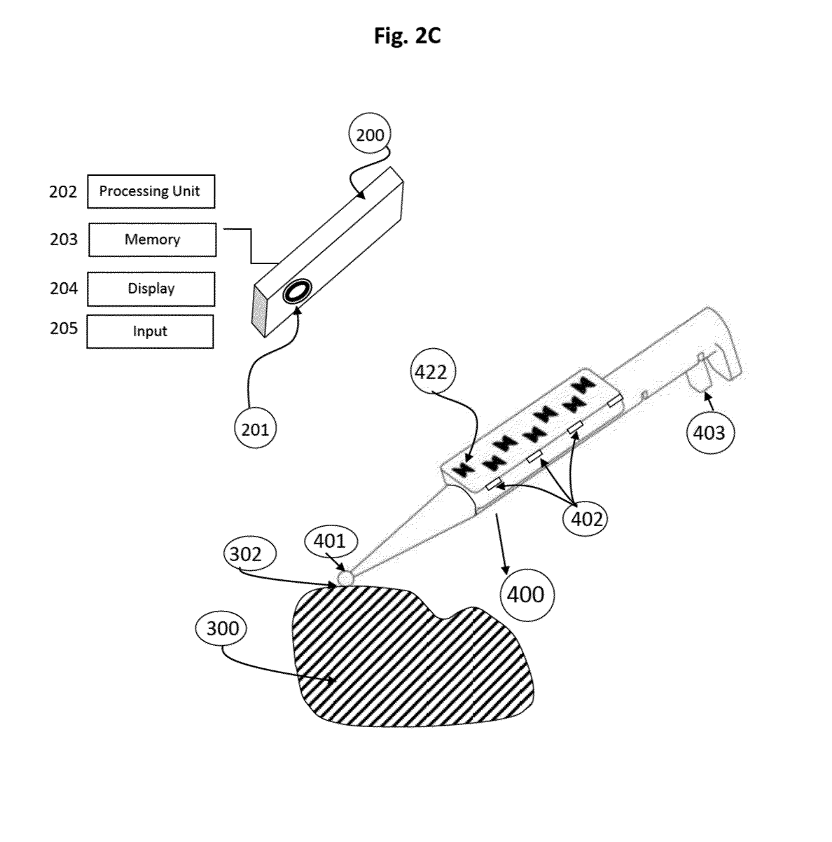

FIGS. 1A, 1B, 2A, 2B and 2C depict the schematic view of systems for surgical tracking.

As depicted in FIGS. 1A, 1B, 2A, 2B and 2C the systems for surgical tracking comprise surgical tracking unit 200 and tracker 100,400.

The surgical navigation/tracking unit 200 comprises at least one sensor 201 which may be an inbuilt digital camera sensor, at least one processor 202, at least one memory unit 203, at least one display unit 204 and at least one input unit 205.

FIG. 1A illustrates the tracker 100 having tracking pattern 120 which is unrecognizable to surgical tracking unit 200 indicating its non-active state, while FIG. 1B illustrates the same tracker 100 having tracking pattern 121 which is recognizable to surgical tracking unit 200 indicating its active state. The tracker 100 may further comprise holding grip 104, a tip 101 to identify the target point such as anatomical landmark 301 for the patient anatomy 300 and sliding mechanism 102 underneath the pattern 120, such that part of pattern is hidden until the trigger 103 is activated.

As shown in FIG. 1B, during surgery, the surgeon provides the input of a specific landmark 301 of the patient's anatomy 300 to the sensor 201 of the surgical tracking unit 200 by pressing the trigger 103. On activation, the sliding mechanism 102 repositions to create a new pattern 121 which is recognizable to the sensor 201 of the surgical tracking unit 200. Once detected, the processing unit 202 and memory 203 of the surgical tracking unit determines the relative location of sensor 200 and the tip 101 of the tracker 100. The surgical tracking unit 200 captures this positional information of tip 100 into its memory 202 which in turn enables it to identify and record specific surgical landmark 301 of the patient anatomy 300.

The recorded information or runtime information about the specific landmark 301 or position of the instrument can be accessed by the surgeon at any instant of time using display 204 and input 205.

FIG. 2A illustrates another tracker 400 with another tracking pattern 420. The sensor 201 of the surgical tracking unit 200 is not able to recognize this pattern 420. FIGS. 2B and 2C illustrate tracker 400 with tracking pattern recognizable to surgical tracking unit 200.

The tracker may comprise a tip 401 to identify the anatomical landmark 301 and sliding mechanism 402 underneath the pattern 420 such that part of the pattern is hidden until the trigger 403 is activated.

As shown in FIG. 2B, the surgeon provides input of a specific landmark 301 of the patient's anatomy 300 to the sensor 201 of the surgical tracking unit 200 by pressing the trigger 403 of the tracker in one direction. On activation, the sliding mechanism 402 of the tracker 400 repositions to create a new pattern 421 which is recognizable to sensor 201 of the surgical tracking unit 200. Upon detection, the surgical tracking unit using its processing unit 202 and its memory 203 determines the relative location of sensor 201 and the tip 401 of the tracker 400.

The surgical tracking unit 200 stores this positional information of tip 401 into its memory 202 which enables it to identify and record the specific landmark 301 of the patient anatomy 300.

In another embodiment, the same tracker 400 can be used to determine multiple landmarks of the patient's anatomy. As shown in FIG. 2C, surgeon provides the input by pressing the trigger 403 in another direction, due to which the sliding mechanism 402 repositions to create a new pattern 422 which is also recognizable to the sensor 201 of the surgical tracking unit 200. Once detected, the surgical tracking unit 200 using its processing unit 202 and memory 203 determines the relative location of sensor 200 and the tip of the tracker 401. The surgical processing unit stores this positional information in its memory and utilizes the same to identify and record another specific landmark 302 of the patients anatomy 300.

This tracker may contain a static component of the pattern and a movable component of pattern, such that when an input is provided by the surgeon using any type of mechanism 102, 402, it moves the movable component to the specified location thereby creating new recognizable pattern.

Tracker pattern 120, 121, 420, 421, 422 can be made of sterile plastic or stickers that can be used only once or can have multiple usage. The pattern can also be made of metal with certain portion etched or embossed or laser marked or processed to create a contrast onto the surface such that it is visible by the sensor of the tracking system. The pattern which is formed by metal or plastic sheet, the tracker comprising at least two layers: an upper layer and a lower layer each bearing a sticker in a manner that upper layer is partially transparent and lower layer is opaque such that when trigger is pressed either said upper layer or lower layer repositions to expose the said lower layer thereby generating new pattern which is recognizable to the surgical tracking unit.

In a hip arthroplasty procedure, alignment of the cup implant that is received by the pelvic bone is very critical. Any incorrect position of cup may lead to dislocation of joint, leg length discrepancy, poor loading condition and thereby resulting in failure of the implant system. Such failure may require expensive and extremely skilled revision surgery by expert surgeons. To accurately place the cup into pelvis, mechanical instruments are preferably used, however the accuracy of these mechanical instruments is limited. Navigation systems do provide some accuracy but can be expensive and cumbersome to use.

The cup placement procedure is divided into two or three stages depending upon the type of implant used. In case of cemented cup implant, the acetabulum is reamed using a reamer as a first step and then a cup is placed along with cement. With regard to an un-cemented press-fit implant, the acetabulum is preferably reamed and then the cup is press-fitted onto the bone using an impaction.

In the present invention, a surgeon identifies and registers the key landmark 301,302 of patient anatomy 300 such as the patient's neutral pelvic plane, acetabulum rim and other key anatomical landmarks in the surgical tracking unit 200 with the help of novel trackers 100, 400. These landmarks are then processed by tracking unit to better understand the patient's anatomy and spatial location relative to the sensor such as camera, this in turn helps to determine the optimal position of the implant for that patient.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-10. (canceled)

11. A system for surgical tracking of a predetermined target in a reference object, the system comprising:
a surgical tracking unit having a sensor, a processing unit, and a memory unit; and
a tracker having a tip to identify at least one target point of the reference object, a plurality of tracking patterns, and an inbuilt mechanism having a trigger, the inbuilt mechanism capable of altering the plurality of tracking patterns,
wherein a first of the plurality of tracking patterns is unrecognizable to the surgical tracking unit, and a second of the plurality of tracking patterns is recognizable to the surgical tracking unit, and
wherein when the tip of the tracker contacts that at least one target point, the trigger is activated to create a new tracking pattern recognizable by the sensor of the surgical tracking unit.

12. The system of claim 11, wherein input of the at least one target point of the reference object is provided by the inbuilt mechanism at any instant to the surgical tracking unit.

13. The system of claim 11, wherein the surgical tracking unit captures positional information of the tip of the tracker in its memory unit by determining the relative location of the tip of the tracker and the sensor in its processing unit to record the at least one target point within a sterile environment.

14. The system of claim 11, wherein only one of the first and second tracking patterns is exposed to the surgical tracking unit at any particular instant of time during surgery.

15. The system of claim 11, wherein the at least one tracker identifies multiple target points by generating the plurality of tracking patterns recognizable to the sensor of the surgical tracking unit with each of the plurality of tracking patterns corresponding to one of the at least one target point.

16. The system of claim 15, wherein the inbuilt mechanism is a sliding mechanism lying beneath each of the plurality of tracking patterns such that when the trigger is pressed, the sliding mechanism repositions to create the new tracking pattern recognizable by the surgical tracking unit.

17. The system of claim 11, wherein the at least one target point of the reference object is a landmark on anatomy of a patient and/or a position of the tip of the tracker in contact with the patient.

18. The system of claim 11, wherein the plurality of tracking patterns is formed by sterile plastic, metal or a combination of both.

19. The system of claim 18, wherein the metal is selected from plain, part embossed, laser marked, or printed, and is processed for creation of a contrasting surface that creates a pattern recognizable by the sensor of the surgical tracking unit.

20. The system of claim 11, wherein the plurality of tracking patterns comprise at least a static component and a movable component, such that when input is provided by the inbuilt mechanism, the movable component of the plurality of tracking patterns is changed thereby creating a new pattern recognizable by the surgical tracking unit.

21. A system for surgical tracking of a predetermined target in a reference object, the system comprising:
a surgical tracking unit having a sensor, a processing unit, and a memory unit; and
a tracker having a tip to identify at least one target point of the reference object, a plurality of tracking patterns, and an inbuilt mechanism having a trigger, the inbuilt mechanism capable of altering the plurality of tracking patterns,
wherein at least one of the plurality of tracking patterns is recognizable to the surgical tracking unit, and
wherein when the tip of the tracker contacts that at least one target point, the trigger is activated to create a new tracking pattern recognizable by the sensor of the surgical tracking unit.

22. The system of claim 21, wherein input of the at least one target point of the reference object is provided by the inbuilt mechanism at any instant to the surgical tracking unit.

23. The system of claim 21, wherein the surgical tracking unit captures positional information of the tip of the tracker in its memory unit by determining the relative location of the tip of the tracker and the sensor in its processing unit to record the at least one target point within a sterile environment.

24. The system of claim 21, wherein only one of the first and second tracking patterns is exposed to the surgical tracking unit at any particular instant of time during surgery.

25. The system of claim 21, wherein the at least one tracker identifies multiple target points by generating the plurality of tracking patterns recognizable to the sensor of the surgical tracking unit with each of the plurality of tracking patterns corresponding to one of the at least one target point.

26. The system of claim 25, wherein the inbuilt mechanism is a sliding mechanism lying beneath each of the plurality of tracking patterns such that when the trigger is pressed, the sliding mechanism repositions to create the new tracking pattern recognizable by the surgical tracking unit.

27. The system of claim 11, wherein the at least one target point of the reference object is a landmark on anatomy of a patient and/or a position of the tip of the tracker in contact with the patient.

28. The system of claim 11, wherein the plurality of tracking patterns is formed by metal selected from plain, part embossed, laser marked, or printed, and is processed for creation of a contrasting surface that creates a pattern recognizable by the sensor of the surgical tracking unit.

29. The system of claim 21, wherein the plurality of tracking patterns comprise at least a static component and a movable component, such that when input is provided by the inbuilt mechanism, the movable component of the plurality of tracking patterns is changed thereby creating a new pattern recognizable by the surgical tracking unit.

30. A system for surgical tracking of a predetermined target in a reference object, the system comprising:
a surgical tracking unit having a sensor, a processing unit, and a memory unit; and
a tracker having a tip to identify at least one target point of the reference object, at least one tracking pattern recognizable to the surgical tracking unit, and an inbuilt mechanism having a trigger, the inbuilt mechanism capable of altering the at least one tracking patten,
wherein when the tip of the tracker contacts that at least one target point, the trigger is activated to reposition a sliding mechanism of the tracker to create a new tracking pattern recognizable by the sensor of the surgical tracking unit.
